(19) United States
(12) Patent Application Publication
(10) Pub. No.: US 2002/0146398 A1
(43) Pub. Date: Oct. 10, 2002

(54) Tissue growth stimulating system

(76) Inventors: Razi Vago, Lehavim (IL); Mordechay (Motti) Beyar, Caesarea (IL)

Correspondence Address: Razi Vago, c/o Disc Orthopaedic Technologies Inc., 101 Interchange Plaza, Cranbury, NJ, 08521, US

(21) Appl. No.: 10/114,438

(22) Filed: Apr. 3, 2002

(30) Foreign Application Priority Data
Apr. 5, 2001 (IL) 142480

(51) Int. Cl. A61K 48/00; A61M 37/00

(52) U.S. Cl. 424/93.21; 424/93.7; 604/93.01

(57) ABSTRACT

The present invention provides method of treatment and device, in cases in which bone growth is involved, as a healing or therapeutical process, such as in spinal fusion procedures, filling of skeletal voids and repair of bone fractures.

After performing the surgical procedure—which includes insertion of bone graft or filling agents, impregnated or not with blood cells or bone marrow—external oxygen and nutrients are supplied by tube directly into the damaged bone area. This method enables the survival of the implanted cells, accelerates bone generation and by that shortens the recovery time.

### BACKGROUND OF THE INVENTION

Bone synthetic chips and bone graft of different kinds and sources are used in cases of bone loss, non union fractures and spinal fusion, in order to fill the voids in the skeleton and encourage the ingrowth of the patient's own bone. Before or during the implantation, the bone synthetic chips or the bone graft can be impregnated with blood cells or bone marrow.

One of the problems in such procedures is lack of blood vessels in the implantation site, which are necessary for the vitality of the implanted living cells within the bone chips or surrounding the grafted material. In intervertebral fusion, for example, the disc area (intervertebral space), wherein the implantation is performed, is poor with blood supply, and necrosis might occur post procedure in tissue placed within the intervertebral space.

In addition, in order to obtain successful intervertebral fusion, the fusion should be achieved as quickly as possible. This requirement is crucial, so as to avoid/minimize the micromovements that occur in time and that might lead to dismantlement of the inserted fusion device or pedicle screws and to pseudoarthrosis.

Indeed, in order to accelerate the bone growth and the intervertebral fusion, it is customary to add to the bone chips factors that stimulate differentiation and bone growth. Nevertheless, these factors do not solve the primary problem of maintaining the implanted cells alive, namely, the poor supply of oxygen and nutrients to the growing process, which is conducted at the end-plate region as well.

The discussed invention in some embodiments thereof provides solutions to both said problems.

### BRIEF SUMMARY OF THE INVENTION

The present invention provides method of treatment and device, in cases in which bone growth is involved, as a healing or therapeutical process, such as in spinal fusion procedures, filling of skeletal voids and repair of bone fractures.

After performing the surgical procedure—which includes insertion of bone graft or filling agents, impregnated or not with blood cells or bone marrow—external oxygen and nutrients are supplied by tube directly into the damaged bone area. This method enables the survival of the implanted cells, accelerates bone generation and by that shortens the recovery time.

### DETAILED DESCRIPTION OF THE INVENTION

The invention relates to a device and a method of treatment, which enables the maintaining of the implanted cells and their adjacent cells alive and promotes bone generation, in bone repair and spinal fusion procedures. This is achieved by supplying, postoperatively, an external flowing medium, which contains, for example, one or more of oxygen, nutrients and bone stimulating factors, as described below.

The Method—Postoperative supply of oxygen and other ingredients to the surgery site by a tubes system, in the area where bone growth is to be achieved.

The Device—The device comprises:

Tubes System: A tube which is introduced into the implantation site, by which the said supply reaches the target area, and a second tube which goes out from the said target area and enables fluid drainage.

Supply Device: A system that supplies the fluid medium with the desired ingredients through the said tubes system.

Regulator: A regulator that regulates the amount and the frequency of the incoming and outgoing fluid and ingredients. The incurrent and excurrent flow should be controlled according to the needs.

In another embodiment of the invention, only one tube is introduced, in which fluid is inserted and removed.

In another embodiment of the invention, only fluid insertion is used. In this case, the amount of fluid is minimal, to prevent massive fluid infiltration to surrounding tissue.

In another embodiment of the invention, one tube is placed inside a second, external tube; One of the tubes, either the internal or the external, is used for the insertion of liquid and ingredients, while the other tube is used for waste drainage.

In such cases the supply system and/or the regulator may also be an implanted one.

The fluid—The fluid could be enriched with oxygen, nutrients, growth and stimulating factors, as well as antibiotic or other ingredients, such as living cells/osteoblasts.

The fluid may flow in a closed circular system, which enables the use of the same medium. If needed, oxygen and other ingredients may be added in controlled amounts and frequencies, and accumulated waste, such as CO₂, Urea and other metabolites, can be drained; Alternatively, the tubes may be disconnected. In this case, the incoming fluid may be renewed, while the outgoing fluid is thrown away, both with controlled amounts and frequencies.

The administration of the said fluid may be continuously or in intervals, as desired during the course of therapy.

The device and its fluid should be kept in sterile conditions.

In the end of the procedure, the said tubes are to be removed from the patient's body.

Having a small diameter tubes, removal of the tubes is performed by a careful pull.

The said invention is not constricted to bone growth but to any living tissue necessitating nutrients or other material to accelerate growth. Such “nutrient” is to be supplied from out of the body through a tubing system, and/or waste can be drained through it as well.

Such invention can be applicable also for regenerating a damaged intervertebral disc by supplying nutrients, oxygen and cells generating disc like properties to such site (through the tubes system or without it). Such cells may be secreting cells, to secrete disc like material, or more specifically, nucleus like material. Such cells also can be cartilage like cells, to proliferate to perform nucleus. Such nucleus shall comply with the physical requirements of an intervertebral disc or artificial disc.

What is claimed is:

1. A method of treatment for maintaining implanted cells alive and accelerate bone growth or cartilage tissue in at least one of bone repair and spinal fusion procedures, including:
providing at least one ingredient from out of the body to a target area.

2. A method of treatment for disc repair according to claim 1, wherein the ingredient comprises living cells into the target area, for at least one of restoring function and achieving function of an affected organ.

3. A method of treatment for bone fusion according to claim 1, wherein the ingredient comprises at least one of living osteoblasts or stem cells into a target area.

4. A method as claimed in claim 1, wherein the ingredient comprises supplying at least one of oxygen, nutrients and growth and stimulating factors.

5. A method as claimed in claim 1, wherein the ingredient comprises at least one of an antibiotic or other drugs.

6. A method as claimed in claim 1, wherein the ingredient is flowed throw a tubular device.

7. A device for supplying ingredients into bone regenerating site, comprising:
a tube which introduces into the regenerated site, and through which at least one of liquid and at least one other ingredient is transmitted to the site (hereafter: “incoming tube”); and
a tube which goes out from the site and drains fluid and waste (hereafter: “outgoing tube”).

8. A device as claimed in claim 7, to which is connected a system that supplies fluid and at least one ingredient into the said incoming tube.

9. A device as claimed in claim 8, including a regulator connected to the said supply system, which is operative to enable regulation and control of at least one of the amounts and the frequencies of the incoming and outgoing liquid and ingredients.

10. A device as claimed in claim 9, wherein at least one of said supply system and said regulator are implanted.

11. A device as claimed in claim 7, wherein said tubing system is organized in a closed circular system, allowing the same medium to flow through it.

12. A device according to claim 11, wherein the ingredient may passes by at least one of osmosis through the tube wall holes and microholes.

13. A device according to claim 7, wherein only one tube is introduced to the implantation site, in which the said fluid and ingredients are inserted and removed.

14. A device according to claim 7, wherein one tube is introduced to the implantation site, in which the said fluid and ingredients are only inserted.

15. A device according to claim 14, wherein minimal amount of fluid is flowed through the tube, to prevent massive fluid infiltration at the surrounding tissue.

16. A device according to claim 7, wherein one tube is positioned inside a second wider tube. One of the tubes, either the external or the internal, is used for insertion of the said liquid and ingredients, while the other is used for waste drainage.

17. A method according to claim 1, wherein said tissue comprises intervertebral disc material.

18. A method according to claim 1, wherein said tissue comprises any living tissue.

19. A device according to claim 7, adapted for at least one of regenerative medicine, other tissues, transplants, as a healing enhancement process regulation and gene delivery systems.
